(19) United States
(12) Patent Application Publication
(10) Pub. No.: US 2011/0136146 A1
(43) Pub. Date: Jun. 9, 2011

(54) BIOASSAY FOR POLYQ PROTEIN

(75) Inventor: Paolo Paganetti, Basel (CH)

(73) Assignee: Novartis AG, Basel (CH)

(21) Appl. No.: 13/057,569

(22) PCT Filed: Aug. 3, 2009
(86) PCT No.: PCT/EP2009/060013

§ 371 Date: Feb. 4, 2011

(30) Foreign Application Priority Data
Aug. 4, 2008 (EP) 08161736.7

(51) Int. Cl. G01N 33/68

(52) U.S. Cl. 435/7.21; 435/7.1; 436/501

(57) ABSTRACT

The present invention relates to bioassays for mutated polyQ protein associated with disease and their use as diagnostic tools, for monitoring disease progression or for monitoring the efficacy of treatment of the disease. In a preferred embodiment the polyQ-protein is polyQ-huntingtin.

### TECHNICAL FIELD

The present invention relates to bioassays for mutated polyQ protein associated with disease and their use as diagnostic tools, for monitoring disease progression or for monitoring the efficacy of treatment of the disease.

### BACKGROUND OF THE INVENTION

A number of diseases are associated with the expression of proteins with polyglutamine repeats, such as Huntington's Disease (HD), spinal bulbar muscular atrophy, several spinocerebellar ataxias and dentatorubral-pallidoluysian atrophy. These disorders are collectively termed ‘polyglutamine diseases’. HD is the most common inherited neurodegenerative disorder with a prevalence of 5 to 8 cases per 100′000. Its main clinical manifestations include motoric dysfunction, psychiatric disturbances and dementia. Numerous symptomatic treatments have been tried for HD without any substantial success (Bonelli and Wenning, 2006) and no approved treatments for HD exist (Bates, 2003). HD is the founding member of the polyglutamine (polyQ) disease family composed of nine autosomal-dominant inherited disorders whose common characteristic is a polyQ-repeat expansion in different ubiquitously expressed proteins (Ross, 2002). The genetic mutation causing HD is a polyglutamine expansion in the huntingtin protein. Expansions beyond 36 glutamines become pathogenic and appear to affect protein folding and successive formation of toxic intracellular fragments and aggregates. The expanded polyQ repeat in the huntingtin gene (Htt) lies in exon 1 and leads to the expression of mutant polyQ-Htt protein (Gusella et al., 1983). The polyQ-repeat expansion may promote a conversion from a native random-coiled to a cylindrical, parallel beta-sheet conformation tethered by hydrogen bonds between the polyglutamine strands (Perutz et al., 1994). Similar to other neurodegenerative disease characterized by protein misfolding like Alzheimer's Disease or Parkinson's Disease, the proteins with helical beta-sheet conformation are prone to form non-soluble protein aggregates (Benzinger et al., 2000).

HD-like symptoms are reversed when expression of mutant Htt is down-regulated in the brain of HD mouse models by RNA interference (DiFiglia et al., 2007) or by tetracyclin-regulated conditional expression (Yamamoto et al., 2000). Interestingly, mutant polyQ- and wild type-Htt are differently metabolized by the cell and display a different pattern of posttranslational modifications (phosphorylation (Warby et al., 2005), proteolytic cleavage (Gafni et al., 2004; Graham et al., 2006; Wellington et al., 2002), cellular localization (Davies et al., 1997; van Roon-Mom et al., 2002) and degradation (Ravikumar et al., 2002). These findings prompted discovery work for HD therapeutics aimed at influencing the misfolding or the clearance of mutant Htt e.g. through upregulation of the chaperone system or induction of the autophagy degradation pathway (Yamamoto et al., 2006). Such approaches may find application for other neurodegenerative diseases caused by protein misfolding.

Currently, there is no bioassay available to assess the effect of such therapies on mutant polyglutamine proteins, e.g. mutant Htt levels in e.g. clinical trials or in therapy.

### SUMMARY OF THE INVENTION

The present invention relates to bioassays for soluble mutated polyQ protein associated with disease and their use as diagnostic tools, for monitoring disease progression or for monitoring the efficacy of treatment of the disease.

In a preferred embodiment the bioassay used is a new homogenous time resolved Förster resonance energy transfer method for Htt detection suitable for high-throughput screening in a neuronal cell line. We show that this “time resolved Förster resonance energy transfer immunoassay” (in the following “Bioassay”) can be modified to quantify endogenous, full-length soluble polyQ-Htt in cellular, animal and human samples. The use of such a bioassay allows for the use of soluble mutant Htt as a marker for disease progression or to monitor the efficacy of drug treatments in preclinical and clinical trials as well as in therapeutic treatment. As the design of the method is highly flexible, it is applicable for use in connection with other diseases, especially diseases associated with other members of the polyQ-family. These proteins are typically expressed intracellularly and assays useful for diagnosis and/or monitoring of disease progression/therapy have not been available until now.

In a preferred embodiment of the present invention the bioassay measures the soluble forms of the polyQ protein, e.g. polyQ huntingtin. In another preferred embodiment of the present invention the bioassay measures the aggregated form of the polyQ protein, e.g. polyQ huntingtin. In yet another preferred embodiment of the present invention the bioassay measures both the soluble and the aggregated form of the polyQ protein, e.g. polyQ huntingtin.

Thus, in one aspect the present invention provides an immunoassay for measuring the amount of the mutated form (polyQ form) of a protein in a biological sample, wherein the protein is selected from the group of huntingtin, androgen receptor, atrophin 1, ataxin 1, ataxin 2, ataxin 3, ataxin 7, TATA box binding protein or alpha1a voltage dependent calcium channel subunit.

In a preferred embodiment of the immunoassay according to the present invention the absolute or relative amount of the corresponding wild-type protein in the sample is additionally measured in the immunoassay.

In a further preferred embodiment of the immunoassay according to the present invention the extent of post-translational modifications of the mutated protein is additionally measured, such as cellular modifications of the expressed protein such as fragmentation by e.g proteolytical cleavage, phosphorylation, acetylation, ubiquitination, SUMOylation, lipid modification or other covalent modifications of the polypeptide backbone.

In a further preferred embodiment of the immunoassay according to the present invention, the immunoassay is a single step assay, i.e. an immunoassay in which no separation or washing is necessary and which can preferably be run after a single biochemical handling. In a further preferred embodiment of the immunoassay according to the present invention, the immunoassay detection technology is based on time-resolved Förster resonance energy transfer or electrochemiluminescence.

In a further preferred embodiment of the immunoassay according to the present invention, the immunoassay detection technology is time-resolved Förster resonance energy transfer. In a further preferred embodiment of the immunoassay according to the present invention, the immunoassay comprises the following steps.
a) contacting the biological sample with a first antibody labeled with a lanthanoide ion cryptate (such as europium or terbium cryptate) and a second antibody labeled with a fluorophore suited for detecting the lanthanide emitted signal, where one of the antibodies is specific for the polyQ part of the mutant protein and the other antibody is specific for a different part of the mutant huntingtin protein and
b) quantifying the amount of mutant polyQ protein in the sample by measuring the fluorescence from the fluorophore by time-resolved Förster Resonance Energy Transfer.

In a further preferred embodiment of the immunoassay according to the present invention, the absolute or relative amount of the corresponding wild-type protein in the sample is additionally measured in the biological sample by additionally contacting it with a third antibody specific for the wild-type form of the protein and labeled with a different fluorophore suited for detecting the lanthanide emitted signal.

In a further preferred embodiment of the immunoassay according to the present invention, the polyQ-protein is polyQ-huntingtin.

In a further preferred embodiment of the immunoassay according to the present invention, the biological sample is derived from the brain, from blood, from muscle or heart or derived from any other peripheral tissue such as skin or hair.

In another aspect of the invention an immunoassay is used for measuring the amount of the mutated polyQ form of a protein in a biological sample, wherein the protein is selected from the group of huntingtin, androgen receptor, atrophin 1, ataxin 1, ataxin 2, ataxin 3, ataxin 7, TATA box binding protein or alphala voltage dependent calcium channel subunit; as a diagnostic tool, for monitoring disease progression or for monitoring the efficacy of treatment of the disease associated with the mutated polyQ form of the protein. The immunoassay is preferably an immunoassay as described in the present application.

### BRIEF DESCRIPTION OF THE FIGURES

FIG. 1: Optimization of Detection Tags, cDNA Constructs and Assay Principle

A. Amino acid sequences of peptides analyzed with the antibody pair beta1 (β1) & 25H10 are shown with the respective epitopes in bold letters. B. Time-resolved FRET analysis of peptides (3 ng/well in duplicates) shown in A. The H1 sequence was selected for tagging mutant Htt and the I6 sequence was selected for tagging wild-type Htt. C. Schemes of cDNA constructs expressed in the inducible HN10 cell lines, indicated are also the antibody binding sites of 25H10, 32A7 and beta1 (β1) at the C-terminal tag. 2B7 is specific for an endogenous amino-terminal epitope of Htt. D. Schematic representation of the protocol used for the cell-based time-resolved FRET assay.

FIG. 2: Detection of Mutant and Wild-Type Htt by time resolved Förster resonance energy transfer

A. Western blot analysis of induced expression of wild-type (573-Q25) and mutant (573-Q72) Htt in HN10 cells compared to that of endogenous full-length Htt. B. After 3 days of induction, the HN10 cell line transfected with mutant and wild-type Htt shows stable Htt expression over time and multiple cell passages. C. Sensitive and specific detection of wild-type or mutant Htt by time-resolved FRET using the indicated antibody combinations after 3 days induction (average value, n=3, standard deviation). D. Optimization of cell lysis conditions for detection of mutant Htt573-Q72 in cells induced for 3 days utilizing the 2B7 & β1 antibody pair (time-resolved FRET measured 50 min after antibody addition, n=6, standard deviation). E. The Z′-factor remained stable between 35 and 110 min after cell lysis and addition of the detecting antibodies although the absolute time-resolved FRET signal increased over time (not shown), induction for 3 days of 573-Q72 HN10 cells (n=6, standard deviation). F. Dose-dependent increase in mutant Htt573-Q72 expression in cells treated with different inducer concentrations (n=6, standard deviation, EC50 ˜250 nM). A-C. the data were generated in 96-well plate format. D-F. the data were generated in 1536-well plate format.

FIG. 3: Schematic representation of the protocol utilized for the Htt bioassay.

Monoclonal antibodies labeled for time-resolved FRET are added by a single pipetting step to cell lysates or tissue homogenates. Simultaneous binding to Htt brings the two antibodies in proximity thus enabling energy transfer from the europium cryptate to the D2-fluorophore. The unique long-lived fluorescence emitted by the lanthanoide cryptate allows for time-resolved measurement and thus temporal distinction from interfering short-lived background fluorescence. As a consequence, accurate Htt determination is possible in strongly colored and autofluorescent biological samples such as blood. B. Scheme of the antibody binding sites on human Htt (tagged Htt573 fragment). 2B7 and MW1 are specific for Htt epitopes located at the amino-terminus and on the polyQ repeat of Htt, respectively. The three monoclonal antibodies Beta1, 32A7 and 25H10 are specific for epitopes added to the carboxy-terminus of the tagged Htt573 fragment. MW1 binding to the polyQ-repeat results in stronger and increased binding to Htt as a function of the polyQ-length (represented by the two antibodies displayed in the figure).

FIG. 4: Mutant huntingtin detection in cellular models of Huntington's disease

A: Time-resolved FRET detection of tagged wild-type (25Q) and mutant (72Q) Htt 573 fragments in induced HN10 cell lysates using the Htt antibody pair 2B7 & MW1. Lysates prepared from non-induced cells served as negative controls. B: Specific detection of induced untagged Htt exon1 fragments in HN10 cells by time-resolved FRET with 2B7 & MW1. C: Quantification of standard amounts of purified recombinant wild-type (25Q) or mutant (46Q) Htt 573 protein by time-resolved FRET with 2B7 & MW1. D. Lentiviral-mediated expression of untagged wild-type (25Q) and mutant (72Q) Htt 548 fragments in ESC-derived neurons analyzed by time-resolved FRET with 2B7 & MW1 revealed that the Htt signal increased as function of polyQ-length although similar Htt expression levels were shown by western blot with 2B7. Htt fragments were not detected in mock infected cells. Tubulin was the loading control. E: Specific detection of endogenous Htt in 140Q-knock-in ESCs. Htt knock-out ESCs (KO) served as negative control. Murine wild-type Htt with 7Q was not detected by 2B7 & MW1. F: Analysis of cell lysates obtained from knock-in ESC-derived neurons expressing endogenous Htt with increasing polyQ-length confirms the polyQ-dependency of the time-resolved FRET assay for Htt. Expression of Htt in the cell lysates shown in this Figure were all confirmed independently by western blot analysis (not shown). All panels show average values of n=3 with error bars indicating standard deviation values.

FIG. 5: Simultaneous determination of wild-type and mutant Htt by duplex time resolved Förster resonance energy transfer

Determination of levels of expression of tamed wild-type and mutant Htt573 in the same sample. HN10 cells expressing both wild-type Htt (25Q) and mutant Htt (72Q) truncated at amino acid 573 and tagged with the beta1 and 32A7 or with beta1 and 25H10 antigens, respectively. Beta1 carried the terbium cryptate mojety that transferred the fluorescent enrgy to 25H10 labelled with D2 as well as to 32A7 labelled with Alexa488. D2 and Alexa488 signals were measured in two different wave length channels.

FIG. 6: Production of pure recombinant Htt protein

SDS poly-acrylamide gel electrophoresis of purified wild-type (Htt573Q25) and mutant (Htt573Q46) protein produced in basteria. Defined amounts of bovine serum albumin (BSA) were loded as a control to estimate the amount of purified Htt obtained in the purification method applied.

FIG. 7: Determination of recombinant wild-type and mutant Htt by the antibody pairs 2B7 & MW1 and 2B7 & 4C9 by duplex time resolved Förster resonance energy transfer

Defined amount of recombinat wild-type Htt (25Q) and mutant Htt (46) were analyzed by duplex time resolved Förster resonance energy transfer using the indicated antibody pairs. Whilst 2B7 & MW1 (Alexa488 channel) recognized better mutant Htt in a polyQ-dependent manner, 2B7 & 4C9 (D2 channel) resulted in a stronger signal for wild-type Htt than for the mutant Htt protein with elongated polyQ.

FIG. 8: Determination of recombinant wild-type and mutant Htt by the antibody pairs 2B7 & MW1 and 2B7 & 2166 by duplex time resolved Förster resonance energy transfer

Defined amount of recombinat wild-type Htt (25Q) and mutant Htt (46) were analyzed by duplex time resolved Förster resonance energy transfer using the indicated antibody pairs. Whilst 2B7 & MW1 (Alexa488 channel) recognized better mutant Htt in a polyQ-dependent manner, 2B7 & 2166 (D2 channel) resulted in a stronger signal for wild-type Htt than for the mutant Htt protein with elongated polyQ.

FIG. 9: Determination of recombinant wild-type and mutant Htt by the antibody pair 4C9 & 2166 by time resolved Förster resonance energy transfer

Defined amount of recombinat wild-type Htt (25Q) and mutant Htt (46) were analyzed by time resolved Förster resonance energy transfer using the antibody pair 4C9 & 2166, which recognized equaly well mutant Htt and wild-type Htt.

FIG. 10: Detection of soluble mutant huntingtin in mice HD models

A: Time-resolved FRET detection of Htt exon1 with 200Q in brains of R6/2 mice. Wild-type mice served as negative controls. The relative concentration of Htt measured by the 2B7 & MW1 antibody pair decreased significantly in 12-week-old symptomatic mice when compared to 4-week-old presymptomatic mice. B: Brain Htt aggregates in R6/2 mice were determined by AGERA (same samples analyzed in A). As expected, a significant increase in aggregate load was found in the older mouse group. C: Specific detection of soluble Htt with the 2B7 & MW1 antibody pair. R6/2 brain homogenates were separated in soluble and insoluble material by ultracentrifugation. The main pool of insoluble Htt aggregates measured by AGERA was recovered in the pellet. In contrast, time-resolved FRET predominantly revealed soluble Htt in the supernatant. D: Detection of Htt in R6/2 tissues. Cortex and muscle samples from 6-week-old R6/2 mice contained significant amounts of Htt when compared to wild-type siblings (n=3). Mutant Htt was detected also in plasma and CSF from 9 to 12-week-old R6/2 mice (n=9) when compared to wild-type mice (n=4). E: Full-length Htt detection in different brain regions of knock-in Hdh140 mice (n=3) expressing Htt with 140Q at endogenous levels. Wild-type siblings (n=3) were used as negative controls. All data average values with standard deviations.

FIG. 11: Analysis of Htt aggregates in mouse brain

A: Representative example of an AGERA blot of brain samples from R6/2 or wild-type (WT) mice at 4 and 12 weeks of age. An age-dependent increase in Htt aggregate load is evident in the R6/2 mouse brain. B: R6/2 brain homogenates of 4 and 12-week-old animals before (Start) and after separation by ultracentrifugation in soluble (Super.) and insoluble (Pellet) material, were analyzed by AGERA. Aggregates are efficiently recovered in the insoluble fraction. No aggregates were detected in the supernatant fractions.

FIG. 12: Detection of Htt in human tissues A: Analysis by time-resolved FRET with 2B7 & MW1 of three post-mortem human HD cortical homogenates (HD) revealed a higher polyQ-dependent Htt signal when compared to three controls (HV); technical triplicates with standard deviation (p<0.001 between HV and HD). B: Box plots of z-transformed relative amounts of Htt measured by time-resolved FRET (technical triplicates) in human snap frozen whole blood samples, EDTA treated erythrocytes and buffy coats isolated from HD (n=5) and control (HV, n=4) subjects. The boxes indicate the range of distribution of 50% of the values, the vertical bars indicate the distribution of all values and the horizontal bars indicate the median values.

FIG. 13: A Principle of time-resolved Förster resonance energy transfer. Excitation of europium-cryptate at 320 nm results in a proximity-dependent, time-delayed FRET emission of the D2-fluorophore at 665 nm. B The emission of Tb²⁺-cryptate is different from Eu³⁺-cryptate and thus in addition to the red D2 acceptor, a green acceptor can be used (Alexa488, fluoresceine). C We spotted two capture antibodies Abeta42 (against the correspondingly tagged wild-type 25Q-huntingtin) and Abeta40 (for mutant 72Q-huntingtin) on a 4 spot/well plate. Only samples containing lysates from cells with induced expression of wild-type 25Q-huntingtin results in a signal when the anti-Abeta42 spot is analyzed. In contrast, the samples containing lysates from cells with induced expression of mutant 72Q-huntingtin are positive in the anti-Abeta40 spots. Htt bound to the plates was detected with the biotinylated Nov1 antibody.

FIG. 14: The assay was used for the analysis of a large group of buffy coats PBMC samples from healthy volunteers and HD patients (kindly provided by Sarah Tabrizi): 100 subjects with two time points for each subject. In this double blinded study, when using a signal threshold established by an earlier experiment using samples provided by Steven Hersch, we correctly assigned 43 our of 44 samples as belonging to the healthy controls. Thus, the assay performs exceptionally well to separate healthy from HD using a blood fraction as starting material.

### DETAILED DESCRIPTION

The present invention relates to bioassays for polyQ protein associated with disease and their use as diagnostic tools, for monitoring disease progression or for monitoring the efficacy of treatment of the disease.

In order that the present invention may be more readily understood, certain terms are first defined. Additional definitions are set forth throughout the detailed description.

The term “mutated polyQ protein” or “polyQ protein” refers to the form of a the polyQ protein containing a polyglutamine expansion associated with the development of disease. For example “mutated polyQ huntingtin” refers to the huntingtin with a polyglutamine expansion beyond 36 glutamines and associated with Huntington's Disease.

The term “single step assay”, refers to an immunoassay in which no separation or washing is necessary and usually can be run after a single biochemical handling.

The term “posttranslational modifications”, refers to cellular modifications of the expressed protein such as proteolytical cleavage, phosphorylation, acetylation, ubiquitination SUMOylation or other covalent modifications of the polypeptide backbone.

The polyglutamine diseases for which the bioassay of the present invention is useful is listed in the following table.

| | | | | | | |
|---|---|---|---|---|---|---|
| | | | Normal | Pathogenic | | |
| Disease | protein | Repeat | repeat length | repeat length | Inclusions | Brain regions most affected |

Typical polyglutamine diseases (gain of function)

| | | | | | | |
|---|---|---|---|---|---|---|
| HD | Huntingtin | CAG | 6-34 | 36-121 | Nucelus and | Striatum, cerebral cortex |
| | | | | | cytoplasm | |
| SBMA | Androgen receptor | CAG | 9-36 | 38-62 | Nucleus and | Anterior horn and bulbar neurons, |
| | | | | | cytoplasm | dorsal root ganglia |
| DRPLA | Atrophin 1 | CAG | 7-34 | 49-68 | Nucleus | Cerebellum, cerebral cortex, basal |
| | | | | | | ganglia, Luys body |
| SCA1 | Ataxin 1 | CAG | 6-39 | 40-82 | Nucleus | Cerebellar Purkinje cells, dentate |
| | | | | | | nucleus, brainstem |
| SCA2 | Ataxin 2 | CAG | 15-24 | 32-200 | Nucleus | Cerebellar Purkinje cells, brain |
| | | | | | | stem, frontotemporal lobes |
| SCA3 | Ataxin 3 | CAG | 13-36 | 61-84 | Nucleus | Cerebellar dentate neurons, basal |
| | | | | | | ganglia, brain stem, sopinal cord |
| SCA7 | Ataxin 7 | CAG | 4-35 | 37-306 | Nucleus | Cerebellum, brain stem, macula, |
| | | | | | | visual cortex |
| SCA17 | TATA box binding | CAG | 4-20 | 20-29 | Nucelus | Cerebellar Purkinje cells, inferior |
| | protein | | | | | olive |

Atypical polygutamine disease (mimicked by missense mutation)

| | | | | | | |
|---|---|---|---|---|---|---|
| SCA6 | Alpha1a voltage- | CAG | 4-20 | 20-29 | Cytoplasm | Cerebellar Purkinje cells, dentate |
| | dependent calcium | | | | | nucleus, inferior olive |
| | channel subunit | | | | | |

Atypical polyglutamine disease (reverse transcription of CTG repeats)

| | | | | | | |
|---|---|---|---|---|---|---|
| SCA8 | Unknown | CTG | 16-34 | >74 | Nucleus | Cerebellar Purkinje cells, granule |
| | | | | | | cells, inferior olive |

The term “antibody” as referred to herein includes whole antibodies and any antigen binding fragment (i. e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., huntingtin). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a VH domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. These antibody fragments are obtained using conventional techniques known to those of skill in the art, and the fragments are screened for utility in the same manner as are intact antibodies.

The phrases “an antibody recognizing an antigen” and “an antibody specific for an antigen” are used interchangeably herein with the term “an antibody which binds specifically to an antigen”.

As used herein, an antibody that “specifically binds to a polyQ protein is intended to refer to an antibody that binds to the polyQ-protein with a K_{D} of 1×10⁻⁸ M or less, 1×10⁻⁹ M or less, or 1×10⁻¹⁰ M or less. An antibody that “cross-reacts with an antigen other than the polyQ-protein” is intended to refer to an antibody that binds that antigen with a K_{D} of 0.5×10⁻⁸ M or less, 5×10⁻⁹ M or less, or 2×10⁻⁹ M or less. An antibody that “does not cross-react with a particular antigen” is intended to refer to an antibody that binds to that antigen, with a K_{D} of 1.5×10⁻⁸ M or greater, or a K_{D} of 5-10×10⁻⁸ M or 1×10⁻⁷ M or greater. In certain embodiments, such antibodies that do not cross-react with the antigen exhibit essentially undetectable binding against these proteins in standard binding assays.

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor syttem such as a Biacore® system.

As used herein, the term “Affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “cross-reactivity” refers to an antibody or population of antibodies binding to epitopes on other antigens. This can be caused either by low avidity or specificity of the antibody or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows chickens, amphibians, reptiles, etc.

Various aspects of the invention are described in further detail in the following subsections.

Time Resolved Förster Resonance Energy Transfer

Time resolved Förster Resonance Energy Transfer (time resolved FRET) is a preferred detection technology for use in a bioassay of the present invention. The technology that has been available for monitoring biomolecular interactions since the early 1990's (Mathis, 1993). There are many different applications of this technology utilizing several aspects of the fluorescence characteristics of lanthanide ions. The large Förster's distance of the rare earth ions is up to 9 nm which is much larger than for many fluorescent compounds which have Förster's distances of between 1-7 nm. The effect of this larger distance is that it is possible to transfer absorbed energy over much longer distances than it is possible for many Förster resonance energy transfer pairs. This then makes it possible to use rare earth chelates as generic immunodetection reagents (Bazin et al., 2001). The second advantage that rare earth Förster resonance energy transfer pairs have is that the time it takes for the fluorescence to decay is greatly delayed thus allowing time resolved fluorescence. The effect of this is to reduce the influence of background fluorescence from small molecules being tested. The ability to monitor ratiometric readouts allows the possibility to correct for liquid dispensing errors, thus helping to reduce assay variability and improve data quality (Imbert et al., 2007).

In time resolved Förster resonance energy transfer (FIG. 13A), energy is adsorbed by a lanthanide-cryptate labeled antibody, lanthanides have an extremely long half-life (Europium and Terbium), complexing to cryptate confers increased stability and the use of a ratiometric measurement allows assay interference correction. Energy is transmitted, when in proximity (with an efficiency of 50% to 95% for distances in the 5-10 nm range), to a second antibody labeled with an appropriate fluorogenic molecule. Historically, the fluorogenic molecule commercially available from CisBio, was XL-665 (a phycobilliprotein hetero-hexameric structure of 105 kDa). XL-665 has an excitation spectrum overlapping that of Eu³⁺-cryptate emission and a maximal light output at 665 nm, a wavelength region Eu³⁺ cryptate only weakly emits light. The second generation D2 acceptor, an organic compound of approximately 1 KDa, is highly compatible with Eu³⁺-cryptate and is now replacing completely the use of XL-665 due to significantly reduced size.

In addition to the Förster resonance energy transfer pair Eu³⁺-cryptate+D2, a second labeling pair has been tested, whereby Lumi4™-Tb, a terbium complex developed by Lumiphore Inc., is the new donor in the time-resolved Förster resonance energy transfer assay. The emission of Tb²⁺-cryptate is different from Eu³⁺-cryptate and thus a green acceptor can be used (Alexa488, fluoresceine). Thus, it is now possible to measure two different proteins (or different combination of epitope, e.g. for analyzing post-translational modifications) in the same sample. The emission of Tb²⁺-cryptate is different from Eu³⁺-cryptate and thus in addition to the red D2 acceptor, a green acceptor can be used (Alexa488, fluoresceine). FIG. 13B

Another preferred detection technology is electrochemiluminescence detection, e.g. as commercially available from Meso Scale Discovery, 9238 Gaither Road, Gaithersburg, Md. 20877, USA.

Meso Scale Discovery (MSD) Technology's electrochemiluminescence detection technology uses SULFO-TAG labels that emit light upon electrochemical stimulation initiated at the electrode surface of microplates. Multiple acceptor antibodies can be spotted in the same well thus allowing detection of up to 10 different proteins in the same sample, bound huntingtin (wild-type and mutant) is detected by a pan anti-huntingtin antibody.

We spotted two capture antibodies Abeta42 (against the correspondingly tagged wild-type 25Q-huntingtin) and Abeta40 (for mutant 72Q-huntingtin) on a 4 spot/well plate. Only samples containing lysates from cells with induced expression of wild-type 25Q-huntingtin results in a signal when the anti-Abeta42 spot is analyzed. In contrast, the samples containing lysates from cells with induced expression of mutant 72Q-huntingtin are positive in the anti-Abeta40 spots. Htt bound to the plates was detected with the biotinylated Nov1 antibody. The result is shown in FIG. 13C.

Thus, theoretically, up to 10 different huntingtin forms (mutant, wild-type, specific post-translational modifications) can be detected in the same sample assuming selective antibodies are available.

Assay Description

Time resolved Förster resonance energy transfer has been used to monitor a number of different biological analytes such as small molecules (e.g. cAMP (Gabriel et al., 2003)) as well as small secreted cytokines (e.g. IL-8 (Achard et al., 2003)) as well as the levels of phosphorylated proteins in in vitro assays (Riddle et al., 2006). There have also been reports of using time resolved Förster resonance energy transfer to monitor the levels of phosphorylated proteins in cell lysates using cell lines over expressing protein substrates of interest. Here we extent these observations by designing combinations of antibodies that give optimal time resolved Förster resonance energy transfer signals and allow the detection of protein expressed at endogenous levels.

### EXAMPLE 1

Technology Development

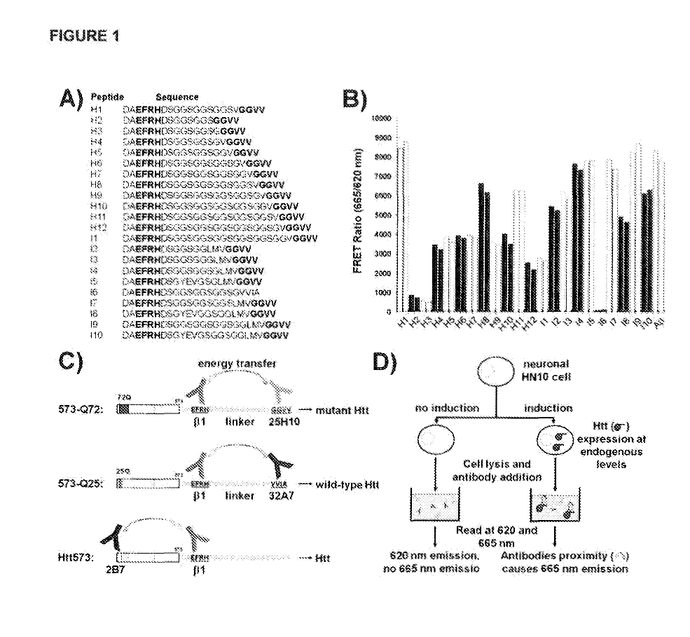

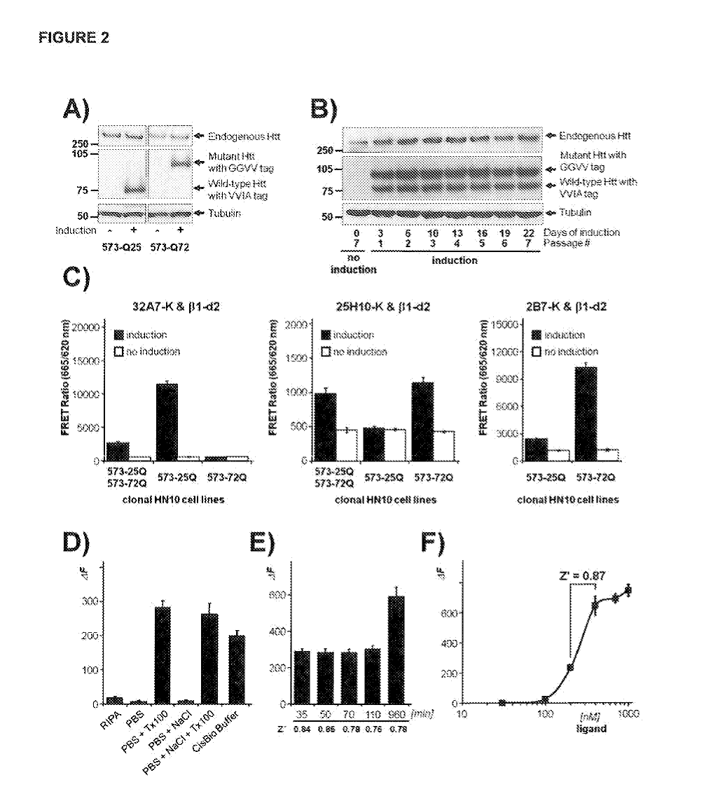

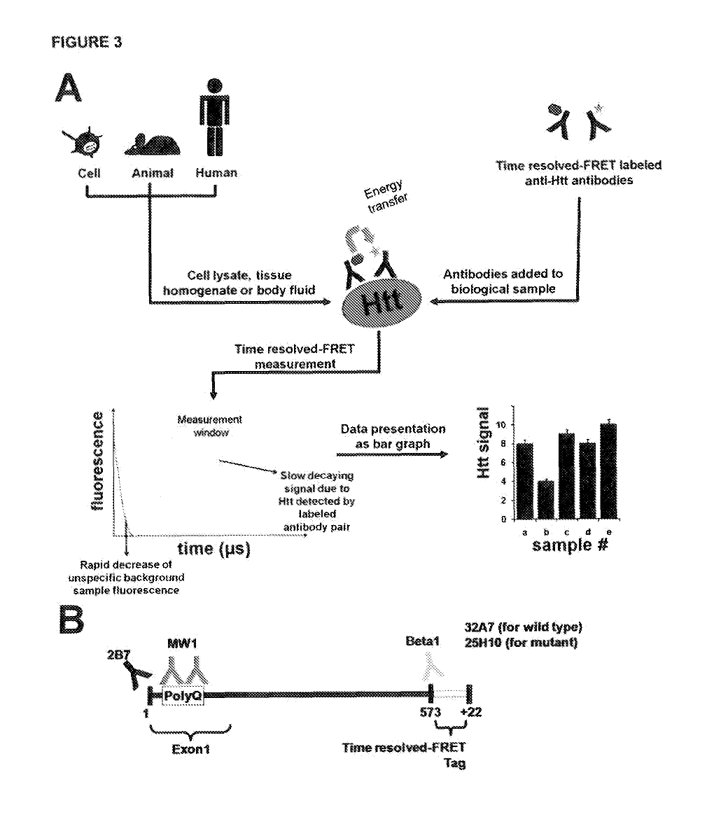

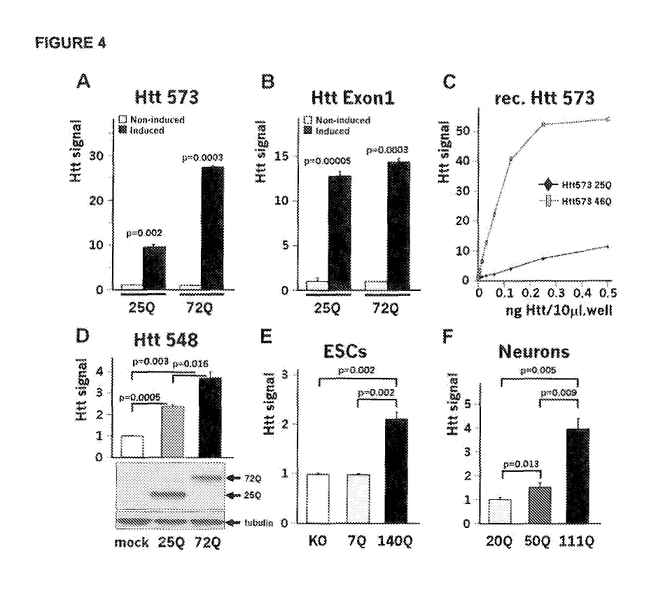

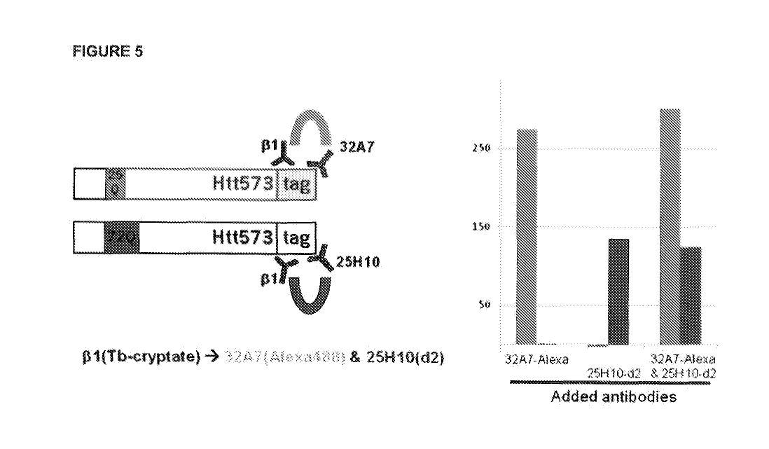

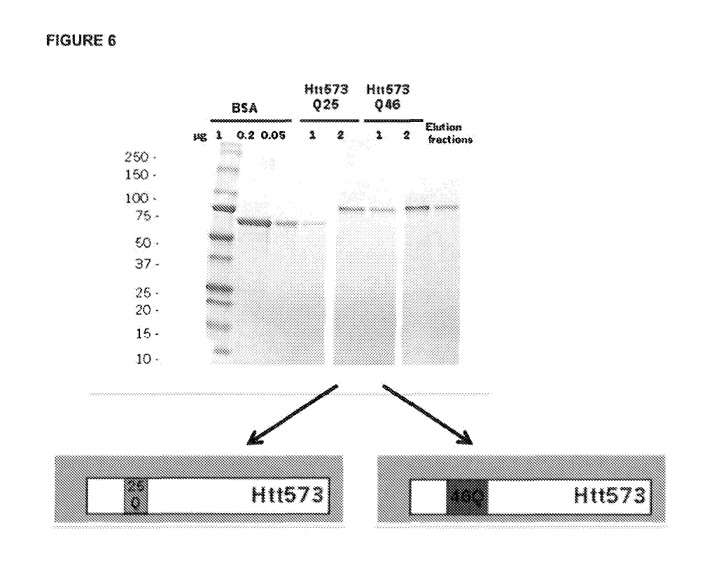

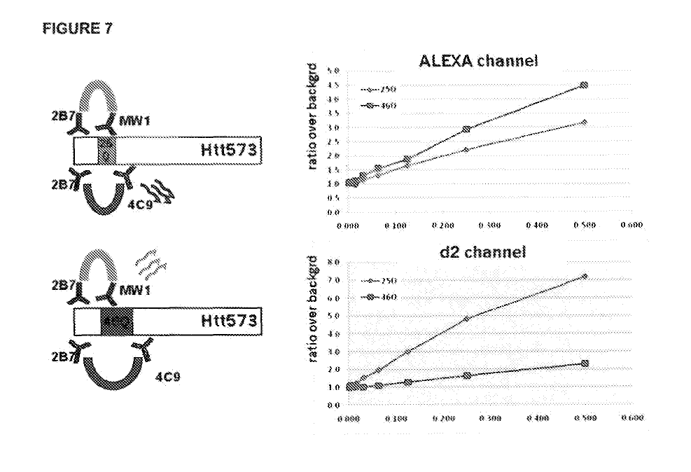

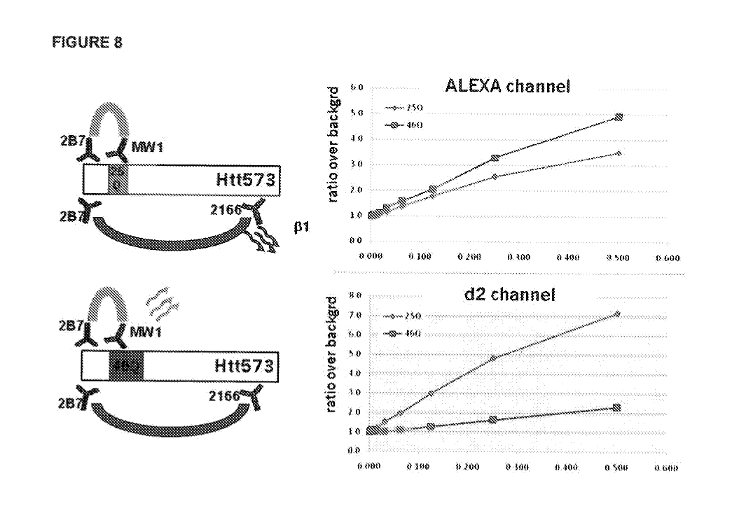

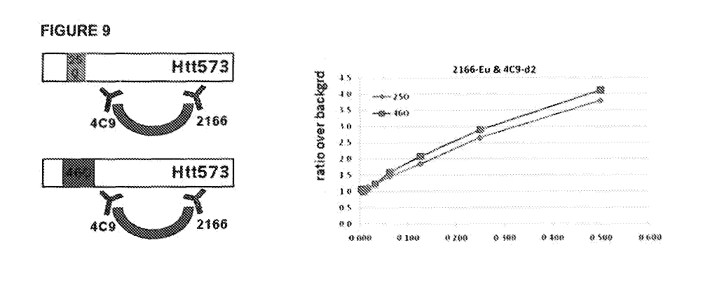

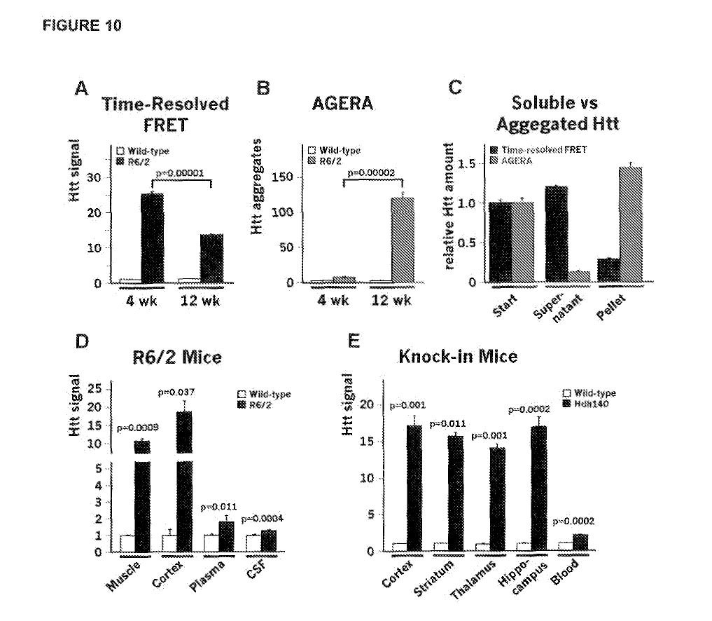

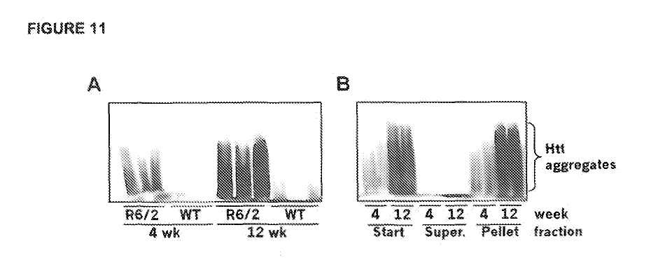

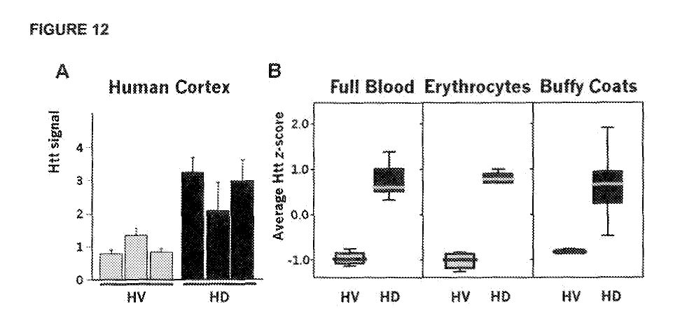

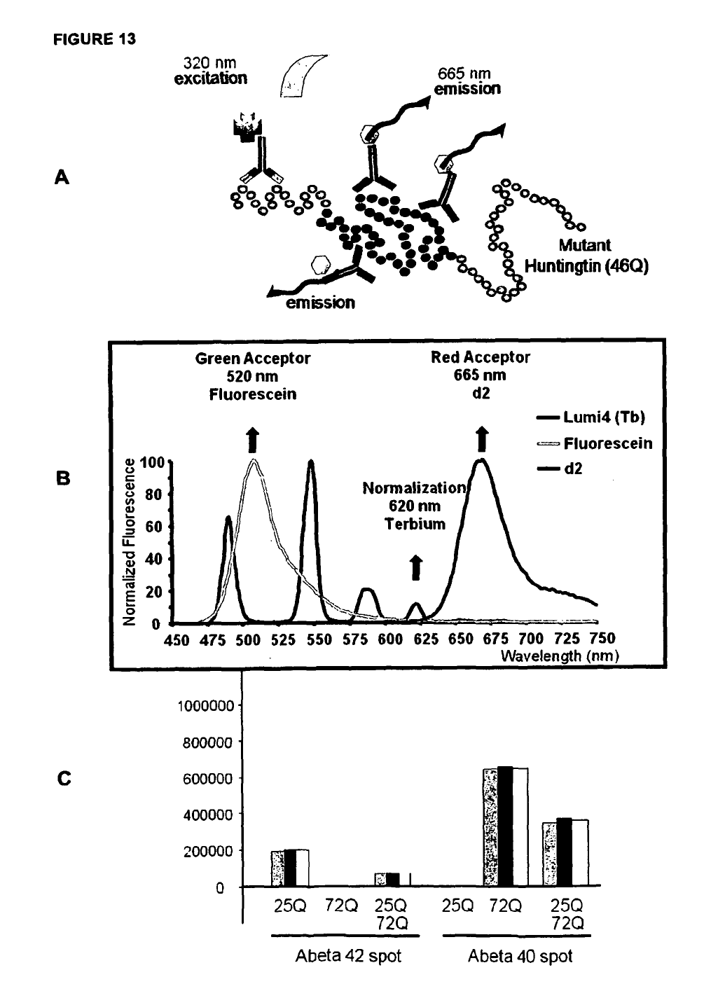

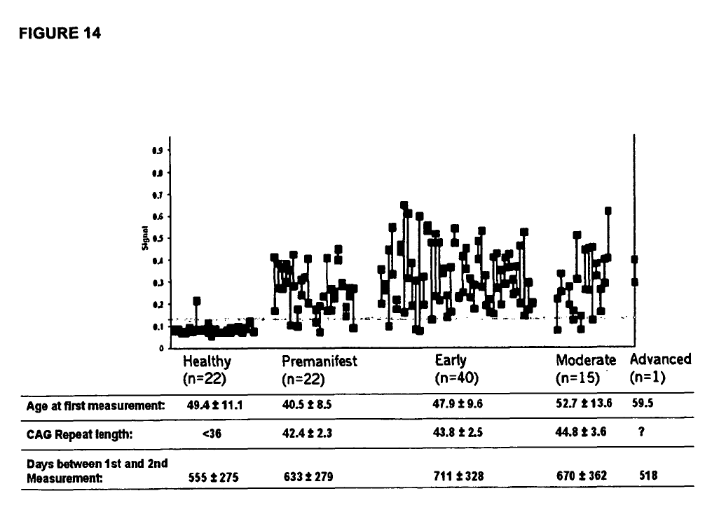

Time resolved Förster resonance energy transfer detection of amyloid β peptide in fluid biological milieu has been previously described (Clarke and Shearman, 2000) and is currently commercially available. The amyloid assay takes advantage of high-affinity antibodies directed against two well characterized epitopes in the amyloid β peptide. We designed a library of small peptides which carry these epitopes (FIG. 1A). Our goal was to use this peptide sequence as a tag for recombinant proteins, making them suitable for time resolved Förster resonance energy transfer detection. Since the efficiency of the Förster resonance energy transfer energy transfer can be influenced by various parameters (Förster, 1948) we tested different peptides with changing linker length and amino acid composition to determine the most suitable peptide sequence. Time resolved Förster resonance energy transfer analysis of purified peptides showed that linker length and sequence of the linker can indeed influence signal intensity significantly (FIG. 1B). For example, peptides with very short linker length (peptides H2 and H3) resulted in low signals probably due to steric hindrance of the two antibodies. Peptide 16 in which the neo-epitope GGVV specific for 25H10 antibody was exchanged for VVIA (specific for 32A7 antibody) failed to result in a signal when using the 25H10 europium labeled antibody, verifying the specifity of the signal. For further experiments we designed two Htt-protein-fragments carrying either the exact H1 peptide sequence as a tag (polyQ-htt/573-Q72) or an alternative sequence in which the neo-epitope GGVV was exchanged for VVIA (WT-htt/573-Q25) (FIG. 1C). We created clonal neuronal HN10 cell lines (Lee et al., 1990) with inducible expression of either polyQ-Htt, wild type Htt or polyQ-Htt and wild type Htt together. These cell lines were subsequently used to establish a cellular high-throughput time resolved Förster resonance energy transfer assay for detection of cellular protein levels (FIG. 1D).

### EXAMPLE 2

Protein Detection and Signal Specificity

The neuronal clonal cell lines created express the tagged polyQ-Htt and wild type Htt at endogeneous levels upon full induction with no detectable basal expression as shown by western blot (FIG. 2A,B). Expression levels of the constructs after induction are stable over time (FIG. 2B). 96-well format experiments showed that highly specific time resolved Förster resonance energy transfer detection of either the WT- or polyQ-Htt-protein in a cellular context is feasible when using the antibody pairs 25H10-K+β1-D2 or 32A7-K+β1-D2 which detect specifically their corresponding tags. In addition, by using 2B7-K antibody specific for an amino-terminal endogenous Htt epitope in combination with the β1-D2 antibody specific for an epitope at the carboxy-terminal tag, allowed for specifically detecting noncleaved, intact Htt-protein levels (FIG. 2C) in the cell lines expressing wild type or polyQ-Htt.

Next, we adapted the assay to a 1536 microwell format. For this, further work was performed using 573-Q72 expressing clone with 2B7-K and β1-D2 antibody combination in order to quantify uncleaved polyQ-Htt-protein. One of the advantages of using a ratiometric readout as used in the time resolved Förster resonance energy transfer method is that adaptation to a miniaturized assay format is readily facilitated because the assay signal is not dependent on the path length of the detection system or the absolute number of particles being detected. In addition ratiometric readout are also more robust to errors in liquid handling again facilitating assay miniaturization. After miniaturizing the format to cells grown directly in a 1536 microwell plate, assay protocol was optimized for lysis buffer (FIG. 2D) and signal development over time (FIG. 2E). Even though induced-to-noninduced signal ratio improved with time of antibody incubation, Z-factors already reached a maximum of 0.86 after shorter incubation periods (FIG. 2E). We proceeded to optimize detection conditions by determining the optimal induction ligand concentration. Induction of polyQ-Htt expression showed good response to changing ligand concentrations, with an IC50 of ˜250 nM (FIG. 2F) and Z-factors of 0.87 between signals at 400 nM and 200 nM inducing ligand, showing the reliability of the assay for a partial, 50% reduction of the polyQ-Htt levels.

### EXAMPLE 3

Time-Resolved Förster Resonance Energy Transfer Assay for Detection of Endogenous Huntingtin.

Amino-terminal fragments of mutant Htt are neurotoxic in vitro and in vivo and are thought to cause Huntington's disease (Arrasate et al., 2004; Li et al., 2000; Varma et al., 2007). Mutant Htt toxicity and aggregation are dependent on polyQ length, Htt fragment length, and level of mutant Htt expression (Colby et al., 2006; King et al., 2008; Machida et al., 2006; Scherzinger et al., 1999; Wang et al., 2005). A critical step towards a disease-modifying treatment or cure for HD is the ability to detect changes in mutant Htt protein levels in the presence of therapeutic modalities in a simple one-step assay. Furthermore, sensitive and efficient Htt measurement may represent a biomarker to analyze the clinical onset or progression of HD. We recently demonstrated the feasibility to measure in one step intracellular mutant Htt using a time resolved-Förster resonance energy transfer assay. In this assay, an antibody pair recognizes a short artificial tag fused to a Htt fragment. (Paganetti et al. 2009)) (FIG. 3). Utilizing such an approach, we developed an assay for wild-type Htt and an assay for mutant Htt using antibody pairs specific for tags located at the carboxy-terminus of the Htt variants i.e. the antibody pairs beta1 & 32A5 and beta1 & 25H10, respectively. Extending the studies with antibodies specific to artificial tags, tagged Htt was readily detected but untagged Htt exon1 constructs were not (FIG. 4). This experiment demonstrated specificity but highlighted that measuring endogenous untagged mutant Htt required an antibody pair directed against endogenous epitopes of Htt.

There is general agreement that Htt toxicity is centered at the amino-terminus of Htt protein, therefore we developed a highly sensitive assay for endogenous mutant Htt using amino-terminal-specific antibodies. The monoclonal antibody 2B7 was conceived as a pan antibody for measuring fragments and full-length mutant Htt and binds to the 17 amino acids immediately amino-terminal to the polyQ-repeat of Htt. When applied in combination with MW1, a polyQ-binding antibody, the 2B7 & MW1 pair specifically detected a 573 amino acid long fragment of wild-type (25Q) and mutant (72Q) Htt expressed in HN10 cells (FIG. 4A), as well as untagged wild-type and mutant Htt exon1 (FIG. 4B). In order to determine the amount of Htt expressed in HN10 cells, we purified recombinant Htt573-25Q protein expressed in bacteria and used it as a standard to spike cell lysates of non-induced HN10 cells in order to calibrate the 2B7&MW1 time-resolved FRET assay (FIG. 4C). We calculated that HN10 cells expressed 0.1 g Htt573-Q25 per mg total protein (0.01% of total HN10 protein) and 0.5 g Htt Exon1-25Q per mg total protein (0.05% of total HN10 protein) and determined a limit of detection in cell lysates corresponding to 25 pM (250 amoles/10 μl per well of a 384 well plate) for the time-resolved FRET assay using the monoclonal antibodies 2B7 and MW1.

A precise comparison of the polyQ-dependent detection of wild-type and mutant Htt in different HN10 cell lines is hampered by clone to clone variations in protein expression levels. As an alternative, we opted for a lentiviral approach for expression of untagged Htt-constructs with 25 or 72 glutamines in a homogenous population of mouse embryonic stem cells (ESC; (Bibel et al., 2004)). A predefined virus titer led to equal expression of the constructs as demonstrated by western blot. Signal strength in time resolved-Förster resonance energy transfer increased with polyQ-length (FIG. 4D). This was expected, since MW1 binds better to expanded polyQ-repeat in mutant Htt when compared to wild-type Htt due to a linear lattice effect (Ko et al., 2001; Li et al., 2007). Also, the use of MW1 in the time-resolved FRET assay may result in a signal not only dependent on protein concentration but also on the affinity as well as on the number of MW1 antibodies simultaneously bound to a long polyQ stretch. In fact, when using same amounts of purified Htt protein with different polyQ lengths spiked into cell lysates of non-induced HN10 cells, we observed a ˜5-fold increase in the signal for Htt573-46Q when compared to Htt573-25Q, measured as the ratio between the slopes calculated from the linear portion of the two standard curves (FIG. 4C). These data demonstrated specific detection of human Htt in a polyQ-dependent manner by the antibody pair 2B7 & MW1 by time-resolved FRET. In view of the polyQ-dependency of the signal and lack of protein standards for each polyQ length, the data in the figures are usually reported as Htt signal over background and the absolute amounts of Htt given in the text only for those samples where the purified protein had the corresponding polyQ length.

For evaluating the use of the time resolved-Förster resonance energy transfer assay to detect endogenous, full-length Htt, we selected ESC with the Htt gene deleted (Htt knock-out as negative control) or modified by a polyQ insertion (polyQ knock-in as positive control). A significant signal was obtained in samples from the 140Q knock-in ESC when compared to samples from the Htt knock-out ESC (FIG. 4E). In contrast, we did not observe a signal above background (mock condition) when using normal ESC. This is consistent with the fact that normal mouse Htt has a polyQ-stretch of only 7 glutamines, evidently too short for recognition by the MW1 monoclonal (Ko et al., 2001). Indeed, in cell lysates obtained from ESC-derived glutamatergic neurons in which varying polyQ-lengths were knocked-in in the endogenous Htt gene, neuronal Htt was detected in a polyQ-length dependent manner (FIG. 4F). Notably, a significant amount of Htt was detected also for 20Q-Htt, a polyQ length representing the majority of the normal human allele. These data showed that single-step time-resolved FRET assay is a bioassay for rapid, quantitative and polyQ-length dependent detection of endogenous Htt protein.

### EXAMPLE 4

Simultaneous Detection of Two Proteins by Duplex Time Resolved Förster Resonance Energy Transfer

One additional application is the duplex determination of tamed wild-type and mutant Htt573 in the same sample, e.g. in HN10 cell lysates expressing both 25QHtt573 and 72QHtt573 whereby the human sequence of Htt was truncated at amino acid 573 and wild-type Htt (25Q) was tagged with the beta1 and 32A7 antigens and mutant Htt (72Q) was tagged with beta1 and 25H10 antigens (FIG. 5, left panel). The beta1 antibody was labelled with Lumi4™-Tb, a terbium complex developed by Lumiphore Inc., which was the new donor in the time-resolved Förster resonance energy transfer assay. The emission of Tb²⁺-cryptate is detected by the red D2 acceptor attached to the 25H10 antibody as well as by the green acceptor Alexa488 (or fluoresceine) attached to the 32A7 antibody. Using two detection channels (red and green) it was possible to measure specifically the amount of wild-type and mutant Htt in the same sample (FIG. 5, right panel) or, theoretically, any other combination of two different proteins or for analyzing two different post-translational modifications of the same protein.

### EXAMPLE 5

Production and Purification of Recombinant Human Huntingtin

A cDNA encoding for a fragment of human Htt 573 amino acid long (with 25 glutamines and truncated after amino acids ThrThrThrGluGlyPro*) was subcloned downstream of Glutathione S-transferase and the vector transfected in E. coli. Bacterial cultures were grown at 37° C. to an OD600 of 1, cooled on ice, isopropyl β-D-1-thiogalactopyranoside was added and cultures were incubated at 12° C. for 18 h. Bacteria were collected by centrifugation and lysed by sonication with phosphate buffered saline and 1% Tween-20. Lysates were cleared by centrifugation and incubated with 2 ml/10 ml lysate Glutathione resin. Beads were washed twice with PBS/0.5% Tween-20 and twice with cleavage buffer (50 mM Tris pH 7.0, 150 mM NaCl, 1 mM EDTA, 1 mM DTT, 0.1% Tween-20, each wash 25× bed volumes). Beads were incubated for 16 h at 4° C. on a rotator with one bed volume cleavage buffer containing 40 U/ml PreScission Protease. The supernatant containing purified Htt protein was recovered and the beads were washed with another bed volume of cleavage buffer (FIG. 6). The same procedure was used to purify the same human Htt fragment with 46 glutamines (FIG. 6). Removal of the glutathione S-transferase by PreScission leaves additional five amino acids (GlyProLeuGlySer) at the amino-terminus of Htt. Protein concentration was determined by measuring the adsorption at 280 nm and confirmed using a commercial protein determination kit.

### EXAMPLE 6

Simultaneous Detection of Two Htt Isoforms by Duplex Time Resolved Förster Resonance Energy Transfer

A second possible application is to study different isoforms of Htt using additional antibodies specific for Htt as listed, but not limited, to the antibodies in the table below:

| Htt | | Epitope |
|---|---|---|
| antibodies | Epitope | sequence |
| Nov1 | Exon1-aa1-17 | MATLEKLMKAFESLKSF |
| 2B7 | Exon1-aa1-17 | MATLEKLMKAFESLKSF |
| 4C9 | Exon1-prolin | QLPQPPPQAQPLLPQPQPPP |
| | reach region | |
| MW1 | Exon1-polyQ | QQQQQQQQQQQQ (>>7Q) |
| MW8 | Exon1-carboxy- | AEEPLHRP |
| | terminus | |
| 2166 | at around amino | SRKQKGKVLLG |
| | acid 444 | |

MW1 and MW8 were generated by Prof. Patterson, Caltech (Ko and Patterson, 2001). 2166 is a commercially available antibody (Chemicon), all other antibodes were generated by our laboratories.

To analyze the specificity of the different antibody pairs, we used purified recombinant Htt protein expressed in bacteria as a standard to calibrate the bioassay and duplex time resolved Förster resonance energy transfer.

In the first application, we used the two antibody pair 2B7 & MW1 and 2B7&4C9 for the detection of recombinant Htt proteins with 25Q and 46Q. We observed that while 2B7 & MW1 (Alexa488 channel) recognized better mutant Htt in a polyQ-dependent manner, 2B7 & 4C9 (D2 channel) resulted in a stronger signal for wild-type Htt than for the mutant Htt protein with elongated polyQ (FIG. 7). As similar result was obtained when 2B7 & 2166 were used instead of 2B7 & 4C9 (D2 channel) in combination with 2B7 & MW1 (Alexa488 channel) (FIG. 8). These data indicated that whenever an antibody pair comprised two antibody recognizing epitopes on opposite site of the polyQ domain of Htt (or the polyQ sequence itself), resulted in polyQ-dependent signal intensity.

Further validating this conclusion, we observed that when using the antibody combination 4C9 & 2166, which recognize two epitpes down-stream of the polyQ stretch, both wild-type Htt (25Q) and mutant Htt (46Q) were detected equally well (FIG. 9). These data demonstrated that it is possible to design a time resolved Förster resonance energy transfer assay for the general detection of multiple isoforms of Htt.

### EXAMPLE 7

Detection of Soluble Mutant Htt in Central and Peripheral Tissues of Murine HD Models and Significant Changes in Soluble Brain Htt as a Function of Disease Progression.

The single-step bioassay for Htt was next used to analyze brain homogenates obtained from 4 and 12 week-old R6/2 mice and aged-matched wild-type mice. R6/2 mice develop an aggressive HD-like phenotype because of the ubiquitous expression of mutant Htt exon1 driven by the human Htt promoter (Mangiarini et al., 1996). FIG. 10 summarizes the data obtained for the HD mice. Robust signals were observed in all transgenic animals analyzed. The mutant Htt specific signal in young, presymptomatic mice was about 25-fold above that measured in wild-type animals (FIG. 10A), which is likely to represent the background noise as endogenous mouse Htt was not detected in ESC Htt-knock out cells (see above). Interestingly, the level of mutant Htt detected in the brain of the older mouse group, which have an advanced HD-like phenotype, was about 45% less than that in young R6/2 mice (FIG. 10A). This decrease in mutant Htt was surprising, as the Htt-aggregate load measured in the same brain samples by AGERA increased as a function of age (Weiss et al., 2008) (FIG. 11A; AGERA blot). One possible explanation for these results was that the time resolved-Förster resonance energy transfer assay using the 2B7-MW1 antibody pair was specific for a mutant Htt fraction distinct from Htt aggregates. To further investigate this possibility, we separated by ultracentrifugation R6/2 brain homogenates into two fractions, one containing only soluble Htt species and one containing Htt aggregates sedimented as an insoluble pellet. Analysis of the supernatant and pellet fractions by AGERA demonstrated successful separation of the insoluble aggregates into the pellet fraction whereby no aggregates were present in the supernatant fractions (FIG. 100 and FIG. 11, AGERA blot). In contrast, we found that the amount of Htt detected by time resolved-Förster resonance energy transfer was predominantly enriched as a soluble material (most likely in a monomeric and oligomeric form) in the supernatant fractions. These data indicated that the time resolved-Förster resonance energy transfer assay was specific for soluble mutant Htt forms. Thus, the decrease in the time resolved-Förster resonance energy transfer signal may indicate recruitment of soluble Htt species into aggregates accumulating as a function of age and disease progression, a mechanism also suggested for other neurodegenerative disorders such as Alzheimer's Disease (Sjogren et al., 2002; Strozyk et al., 2003).

We extended our analysis to include muscle and plasma samples from 6 week old as well as corticospinal fluid samples from 9 to 12 old R6/2 or WT mice. We found significant detectable amounts of mutant Htt in the R6/2 mice when compared to their normal siblings in all tissue samples analyzed, although the signals were significantly lower than those detected in cortical extracts (FIG. 10D).

In the context of a bioassay for mutant Htt, the R6/2 mouse model of HD based on the expression of a short fragment of Htt may have only a limited value as a model of the human situation in which mutated full-length Htt is expressed. As an alternative, we applied the time resolved-Förster resonance energy transfer assay for the analysis of mutant Htt in a knock-in mouse model expressing endogenous mouse full-length Htt with a polyQ-stretch of 140 glutamines (Menalled et al., 2003). Similar to the R6/2 mouse samples, significant amounts of mutant Htt were detected in every brain area analyzed as well as in full blood samples (FIG. 10E) obtained from the polyQ-knock-in mice.

### EXAMPLE 8

Sensitive and PolyQ-Dependent Huntingtin Detection in Human Tissue Samples.

As the sensitivity and specificity of the mutant Htt bioassay was clearly demonstrated in mouse tissue samples, we next turned our attention to detection of endogenous mutant Htt in human post-mortem cortex tissue obtained from three healthy volunteer (HV) controls and three HD brains. In a first experiment, the Htt bioassay measured a 2.7-fold higher level of Htt (p<0.001) in all three HD patients compared to the HV controls (FIG. 12A), demonstrating the utility of the assay for sensitive, rapid and polyQ-dependent determination of Htt in a human tissue. Availability of recombinant Htt proteins with polyQ length corresponding to the normal (25Q) and to the great majority of the HD alleles (460) [37], allowed us to estimate an Htt concentration in the human HD cortex amounting to ˜20 ng/mg total protein (˜10 nM), both for the normal as well as for the mutant Htt protein.

As in most clinical settings it is much easier to obtain blood samples, we then tested three different blood fractions obtained from five living HD patients and four HV subjects. In a blinded experiment, it was possible to unambiguously distinguish the HD patient samples from the four control samples in whole blood, isolated erythrocytes and buffy coats. The relative amount of Htt determined by the time-resolved FRET assay was z-transformed to allow score comparison between the blood fractions (FIG. 12B). When comparing the average z-scores for each subject across the tissues, the repeated-measures ANOVA demonstrated a significant group effect (F(1,7)=61.07; p<0.001, partial η2=0.90). Beside the expected significant difference in total function capacity (TFC), no other effect or interaction reached significance. As an example, although different between the groups, gender did not explain the difference between HD and HV. When analyzing the five HD samples, no significant linear correlation between Htt concentration and disease progression (TFC) was reached, although we observed a promising trend in whole blood (R=0.50) and buffy coats (R=0.65) but not in erythrocytes (R=0.03). The box plot in FIG. 12 shows no overlap between the HD and the HV samples, clearly demonstrating that the Htt bioassay efficiently identifies endogenous mutant Htt in tissue samples from HD patients and may complement DNA-based genotyping.

### EXAMPLE 10

### Longitudinal Analysis of Human Buffy Coat Samples.

Towards routine use for clinical research projects, the assay was used for the analysis of a large group of buffy coats PBMC samples from healthy volunteers and HD patients (kindly provided by Sarah Tabrizi): 100 subjects with two time points for each subject. In this double blinded study, when using a signal threshold established by an earlier experiment using samples provided by Steven Hersch, we correctly assigned 43 our of 44 samples as belonging to the healthy controls. Thus, the assay performs exceptionally well to separate healthy from HD using a blood fraction as starting material. The results are shown in FIG. 14.

We have developed a bioassay for determination of soluble mutant Htt and demonstrated its use to measure mutant Htt levels in cell lysates, animal and human tissues.

The time resolved-Förster resonance energy transfer assay is a simple, one-step methodology that requires only small sample volumes. Quantitative determination of mutant Htt levels are therefore possible with as little as 5 μl human full blood, providing the possibility to determine soluble mutant Htt levels multiple times over a longer clinical trial period without affecting the patient as obtaining the sample is minimally invasive. In addition, the ability to correct for artifacts using the time resolved-Förster resonance energy transfer method allows for a very reliable quantification of mutant Htt even in small sample sizes (Imbert et al., 2007).

To verify the specifity of our detection method for soluble mutant Htt, several experimental steps were taken. Importantly, we based our bioassay on a method that has been recently described to detect intracellular levels of tagged Htt fragments in a sensitive, robust and reliable manner as indicated by a high Z-factor value, a common statistical parameter that reflects assay quality in terms of reliability and robustness (Zhang et al., 1999), (Weiss et al. submitted). By exchanging the detection antibodies of the high-throughput screen to an antibody pair that recognizes endogenous Htt epitopes, we were able to show detection of untagged Htt fragments in a stable neuronal cell line with inducible expression of tagged Htt. In order to specifically detect mutant Htt levels over wild-type Htt, one of the antibodies is directed against the polyQ-repeat that is elongated in Huntington's Disease. We then showed that the signal intensity directly correlates with the polyQ length in lentiviral infected embryonic stem cells as well as in cell lysates obtained from wild-type and polyQ-knock-in embryonic stem cells and embryonic stem cell derived neurons. Critically, using knock-out embryonic stem cell lysates void of any Htt protein expression, we were able to prove the Htt specificity of our signal. As the detection method is polyQ-length dependent, it should be noted that while wild-type Htt is not detected in murine cells or animal tissue due to the WT-polyQ-length of only 7 glutamines, human healthy polyQ length normally resides around ˜20 glutamines (Myers, 2004), a length that is also detected by our method. However, the intensity of this healthy Htt derived signal only contributes little to the total signal that is largely comprised of mutant polyQ Htt detection with polyQ-length >39 glutamines.

We proceeded to analyze mouse models of HD and for the first time were able to quantifiably determine soluble, non-aggregated mutant Htt levels in various tissue samples of two different HD mouse models. Notably, we found that the levels of soluble mutant Htt decrease while the amount of insoluble Htt aggregates increase in the brains of aging R6/2 mice. This decrease of an aggregation prone monomeric species upon aging resembles similar findings in Alzheimer Disease whereby a decrease in Abeta42 levels is a marker for disease progression (likely to be caused by recruitment of soluble Abeta as a function of increased plaque burden).

Finally, we tested human post-mortem cortex samples as well as full blood and blood-derived human samples from living control and HD patients. We were able to clearly distinguish between healthy and HD patient samples by the intensity of the signal alone. This quantitative detection of soluble Htt in readily available human tissue sample opens up the possibility to determine the value of this soluble mutant Htt quantification for use as a potential biomarker for HD disease progression. In that regard, it is interesting that the signal measured in buffy coat fraction trends to correlate with severity of disease progression as determined by the total function capacity score in the analyzed HD patients. This trend was not observed in full blood or erythrocytes. However, erythrocytes represent the vast majority of cells found in full blood and since erythrocytes display a shorter lifespan than some of the lymphocytes found in the buffy coat fraction, this difference could be due to the longer lifespan of a lymphocytes subpopulation in which effects of mutant huntingtin monomer expression, e.g. huntingtin aggregation, can accumulate over time, leading to a decreased signal of soluble mutant huntingtin similar to what we observed in R6/2 mice with advanced disease progression. Further longitudinal studies with a larger HD patient population could help to elucidate this intriguing possibility. In addition, since potential HD therapies could be aimed at influencing the soluble mutant Htt pool directly (e.g. compounds that alter aggregation, compounds that act on the chaperone system or compounds that act on autophagy) the precise quantification of soluble mutant Htt could also find application as a marker for treatment success in human clinical trials.

In summary, our bioassay is a very simple, one-step methodology that requires small sample volumes. In addition, the artifact corrected nature of time resolved-Förster resonance energy transfer allows for very reliable Htt-quantification with a single small sample per subject, making the method useful for experiments that are limited by sample numbers or sample volume as it is often found in human clinical trials. Because signal specifity of the method depends on the antibody pair used, the method could also find further application not only for HD but also for other diseases, especially other polyQ-diseases like the spinocerebellar ataxias.

An interesting development of our assay is multiplexing and thus allow to establish relative measurements for several huntingtin forms. We have demonstrated that this is possible for mutant versus normal huntingtin, but it will be very interesting to develop additional antibody combinations to measure the extend of post-translational modifications relative to normal huntingtin such as proteolytical cleavage, phosphorylation, acetylation, ubiquitination SUMOylation and other naturally occurring covalent modifications of the polypeptide backbone.

The sample material for use in an assay according to the present invention may be human body fluids such as urine, saliva, plasma, serum and corticospinal fluid as well as tissue extracts from organs such as brain, muscle, skin, hair, blood cells and other central and peripheral human organs. Tissue extracts can be obtained by homogenization or detergent lysis of tissue biopsies

When the bioassay of the present invention is used as a diagnostic tool any of the human samples described above can be analysed and compared to those obtained from healthy volunteer controls.

When the bioassay of the present invention is used for monitoring disease progression any of the human samples described above can be analyzed longitudinally as a function of disease progression or before and after phenoconversion

When the bioassay of the present invention is used for monitoring the efficacy of treatment of the disease any of the human samples described above can be analyzed before and after pharmacological treatment

Methods & Materials

Generation of Peptides and Antibodies

Peptides comprising epitopes, against which antibodies 25H10, 32A7 or β1 are reactive, and which are separated by different linker sequences, were custom produced by MIT biopolymers laboratory. Amyloid β40 peptide was purchased from Bachem (Bubendorf, Switzerland).

25H10 antibody specific against GGVV-epitope, 32A7 antibody specific against VVIA and β1 antibody directed against EFRH are described elsewhere (Paganetti et al., 1996). 2B7 antibody was custom designed against the first 17 amino acids of Htt protein (GENOVAC, Freiburg, Germany). MW1 antibody specific against the polyglutamine stretch of Htt and developed by Dr. Paul Patterson were obtained from the Developmental Studies Hybridoma Bank developed under the auspices of the NICHD and maintained by The University of Iowa, Department of Biological Sciences, Iowa City, Iowa 52242. Custom europium cryptate and D2-fluorophore labeling of the antibodies were performed by CisBio (Bagnols/Ceze, France). Depending on the batch used, antibodies were cross-linked to 5 to 7 mol europium cryptate or D2-fluorophore per mol antibody.

Generation of Neuronal Cell Lines

Neuronal HN10 cells (Lee et al., 1990) were used to create inducible clones with expression of 573-Q25 and/or 573-Q72 Htt amino-terminal. In short, cells were transfected with the rheoswitch receptor plasmid (New England Biolabs) and cultured under selection of 1 mg/ml G418 (Invitrogen). Clones were screened for cell morphology, transfected with inducible luciferase reporter construct and induced for 2 days. Clone with best induction ratio were selected and used for subsequent transfection with 573-Q25 or 573-Q72 inducible plasmid. After selection with 1 mg/ml G418 and 1 mg/ml Hygromycin (Invitrogen), inducible expression of Htt fragments in the clonal lines were monitored with herein described time resolved Förster resonance energy transfer detection method and clones with no basal expression and highest inducible expression were chosen for use in assay format.

Other Cellular Models.

The knock-in embryonic stem cells (ES cells) were generated as described in (Wheeler et al., 1999; White et al., 1997). The neomycin selection cassette was removed by a second electroporation with a plasmid expressing cre recombinase. Embryonic stem cell-derived neurons (ES neurons) were generated using the differentiation protocol as published by (Bibel et al., 2007; Bibel et al., 2004). In brief, ES cells were cultivated on mitomycine-inactivated mouse embryonic fibroblasts for at least two passages after thawing in ES medium containing 15% foetal calf serum (FCS) and 1000 U/ml LIF (leukemia inducing factor). Subsequently, they were cultured without fibroblast feeder cells for two more passages. Embryoid bodies (EBs) were formed on bacterial dishes in EB medium containing 10% FCS but no LIF and incubated for 8 days with the addition of retinoic acid on the last four days. EBs were dissociated by trypsinisation and plated on poly-l-lysine and laminin coated plates in N2 medium and changed to neuronal differentiation medium as described by (Brewer and Cotman, 1989) two days after dissociation.

Detection of Peptides by Time Resolved Förster Resonance Energy Transfer

Peptides were prediluted in DMSO to 800 μg/ml. DMSO solutions were further diluted in ⅕ RIPA buffer to 3 ng/ml final concentration. 3 ng/ml amyloid β40 peptide was used as control. 10 μl peptide solution per low-volume 96-well were mixed with 5 μl of antibody solution (beta1-D2 20 ng/well, 25H10-K 2 ng/well in 50 mM NaH₂Pa₄, 400 mM NaF, 0.1% BSA, 0.05% tween) and incubated at 4° C. overnight. 620 and 665 nm signals were measured with a RUBYstar (BMG Labtech) reader.

96 Well Format

20.000 cells/well were seeded in 100 μl normal grow medium (DMEM (Gibco), 10% FCS, penicillin and streptomycin). After 2 h medium was removed and 200 μl inducing medium (normal growth medium plus inducing ligand) was added to start expression of Htt fragments. After 3 days, medium was removed and 30 μl/well readout buffer (20 μl of different lysis buffers and 10 μl of β1-D2 and 25H10-K or 32A7-K in 50 mM NaH₂Po₄, 400 mM NaF, 0.1% BSA, 0.05% tween) was added. After incubating 30 min incubation at room temperature, lysates were transferred to low volume black bottom 96 well plate. After 3 h at 4° C. 620 and 665 nm signals were measured. with a RUBYstar (BMG Labtech) reader.

1536 Well HTS Miniaturization and Compound Screen

573-Q72 expressing clone was incubated for 72 hours at 37° C., 5% CO₂ with inducing medium to facilitate expression of polyQ-htt construct. 3 μl of a 2000 cells/μl cell suspension were then seeded per well in a 1536-microtiterplate (Greiner) and incubated overnight −/+ compound treatment. 3 μl of Lysis buffer (1× PBS+1% Triton X-100, complete protease inhibitors) were added and incubated for 30 min at room temperature. 2 μl antibody dilution in 50 mM NaH₂PO₄, 400 mM NaF, 0.1% BSA, 0.05% tween was added to a final dilution of 60 pg/well europium labeled antibody and 800 pg/well D2-labeled antibody. Plates were incubated at room temperature as indicated. Measurements were performed with a View Lux machine with the following settings: Label 1 time resolved Förster resonance energy transfer_Eu-K_(E:800K,Xsec,BF4, GN:high,SP:slow), Label 2 time resolved Förster resonance energy transfer_XL665_(E:800K,Xsec,BF4, GN:high,SP:slow)

Animal Models.

Heterozygous transgenic R6/2 males of CBAxC57BL/6 strain were obtained from G. Bates laboratory (Mangiarini et al., 1996) and bred with CBAxC57BL/6 F1 females. The offspring were genotyped by PCR assay of DNA obtained from tail tissue. The animals were housed in a temperature-controlled room that was maintained on a 12 hr light/dark cycle. Food and water were available ad libitum. All experiments were carried out in accordance with authorization guidelines for the care and use of laboratory animals.

For time resolved Förster resonance energy transfer assay detection of huntingtin, 2-3 months old animals were anesthetized with 3-5% isofluran followed by an intraperitoneal dose of 100 mg/kg Ketamin and 10 mg/kg xylazine. After CSF and blood collection, animals were given a sodium pentobarbital overdose (150 mg/kg). Muscle (gastrocnemius) and brain were immediately further collected for Förster resonance energy transfer analysis. Brain homogenate loaded 100 ug per well (10 mg/ml protein concentration)

Aggregate Analysis (AGERA).

AGERA analysis was performed as described (Weiss et al., 2008). In short, R6/2 brains were homogenized in 10 volumes (w/v) PBS+0,4% TritonX100 and Complete Protease Inhibitor (Roche Diagnostics). Brain samples corresponding to 0.15 □g of total protein were loaded per AGERA lane. For separation of brain homogenates into soluble and non-soluble fractions, homogenates were centrifuged at 124 000 g for 1 h, supernatant was aliquoted (soluble fraction) and pellet was resuspend in equal to starting volume PBS+0.4% TritonX100 (non-soluble fraction).

BioAssay

Brain and muscle tissue were homogenized in 10× volume sample buffer (PBS+1% Triton X-100+ compleate protease inhibitor). Blood, plasma and corticospinal fluid samples were prediluted 1:1 in sample buffer. 10 μl sample and 5 μl antibody dilution (europium cryptate and D2 labeled antibodies in 50 mM NaH₂PO₄, 400 mM NaF, 0.1% BSA, 0.05% Tween) was added to each well to a final dilution of 1.5 ng/well 2B7-europium labeled antibody and 30 ng/well MW1-D2-labeled antibody. Plates were incubated at 4° C. for 1 h. Measurements were performed with a Xenon-lamp Envision Reader for 620 and 665 nm wavelengths after excitation at 320 nm (time delay 100 μs, window 400 μs, 100 flashes per well).

Data Analysis

Time resolved Förster resonance energy transfer measurement results in two different signals. 620 nm signal from the europium cryptate labeled antibody can be used as an internal reference for possible interfering artifacts of the assay such as signal quenching or absorption by compounds, sample turbidity as well as differences in excitation energy or sample volume. 665 nm signal results from D2 labeled antibody which is excited by time resolved energy transfer from the europium cryptate. The calculated 665/620 nm ratio therefore is an artifact corrected specific signal of the two bound antibodies to their antigen and hence a precise reflection of the amount of antigen present in the sample. For 96 well data time resolved Förster resonance energy transfer signals are given as the ratio between those two wavelengths:

Ratio_{665/620induced}−Ratio_{665/620noninduced}*10000

For 1536 microtiter well optimization data, time resolved signals are presented as ΔF values, a format more suitable to take day-to-day assay variations into account as it is a background correctd value:

ΔF=(Ratio_{665/620induced}−Ratio_{665/620noninduced})/Ratio_{665/620noninduced}*100

Analysis of high throughput screening data was conducted using an inhouse data analysis software, this software is able to normalize activity to % remaining activity with the use of high and low control samples present on a plate and to correct plate effects using a local regression algorithm that corrects for local plate effects (Gubler, 2006). Z-factor was calculated according to (Zhang et al., 1999).

Statistical Analysis

Quantification of cellular and mouse values are presented as averages with standard deviations. Significances were calculated by students' t-test.

### REFERENCES

Achard, S., Jean, A., Lorphelin, D., Amoravain, M. and Claret, E. J. (2003). Homogeneous assays allow direct “in well” cytokine level quantification. Assay Drug Dev Technol 1, 181-5.

Arrasate, M., Mitra, S., Schweitzer, E. S., Segal, M. R. and Finkbeiner, S. (2004). Inclusion body formation reduces levels of mutant huntingtin and the risk of neuronal death. Nature 431, 805-10.

Balbirnie, M., Grothe, R. and Eisenberg, D. S. (2001). An amyloid-forming peptide from the yeast prion Sup35 reveals a dehydrated beta-sheet structure for amyloid. Proc Natl Aced Sci USA 98, 2375-80.

Bates, G. (2003). Huntingtin aggregation and toxicity in Huntington's disease. Lancet 361, 1642-4.

Bates, G. P. and Hockly, E. (2003). Experimental therapeutics in Huntington's disease: are models useful for therapeutic trials? Curr Opin Neurol 16, 465-70.

Bazin, H., Preaudat, M., Trinquet, E. and Mathis, G. (2001). Homogeneous time resolved fluorescence resonance energy transfer using rare earth cryptates as a tool for probing molecular interactions in biology. Spectrochim Acta A Mol Biomol Spectrosc 57, 2197-211.

Benzinger, T. L., Gregory, D. M., Burkoth, T. S., Miller-Auer, H., Lynn, D. G., Botto, R. E. and Meredith, S. C. (2000). Two-dimensional structure of beta-amyloid(10-35) fibrils. Biochemistry 39, 3491-9.

Bibel, M., Richter, J., Lacroix, E. and Barde, Y. A. (2007). Generation of a defined and uniform population of CNS progenitors and neurons from mouse embryonic stem cells. Nat Protoc 2, 1034-43.

Bibel, M., Richter, J., Schrenk, K., Tucker, K. L., Staiger, V., Korte, M., Goetz, M. and Barde, Y. A. (2004). Differentiation of mouse embryonic stem cells into a defined neuronal lineage. Nat Neurosci 7, 1003-9.

Bonelli, R. M. and Wenning, G. K. (2006). Pharmacological management of Huntington's disease: an evidence-based review. Curr Pharm Des 12, 2701-20.

Brewer, G. J. and Cotman, C. W. (1989). Survival and growth of hippocampal neurons in defined medium at low density: advantages of a sandwich culture technique or low oxygen. Brain Res 494, 65-74.

Clarke, E. E. and Shearman, M. S. (2000). Quantitation of amyloid-beta peptides in biological milieu using a novel homogeneous time-resolved fluorescence (HTRF) assay. J Neurosci Methods 102, 61-8.

Clegg, R. M. (1996). Fluorescence resonance energy transfer. In Fluorescence Imaging Spectroscopy and Microscopy, (ed. X. F. W. a. B. Herman), pp. 179-252: John Wiley & Sons.

Colby, D. W., Cassady, J. P., Lin, G. C., Ingram, V. M. and Wittrup, K. D. (2006). Stochastic kinetics of intracellular huntingtin aggregate formation. Nat Chem Biol 2, 319-23.

Davies, S. W., Turmaine, M., Cozens, B. A., DiFiglia, M., Sharp, A. H., Ross, C. A., Scherzinger, E., Wanker, E. E., Mangiarini, L. and Bates, G. P. (1997). Formation of neuronal intranuclear inclusions underlies the neurological dysfunction in mice transgenic for the HD mutation. Cell 90, 537-48.

DiFiglia, M., Sena-Esteves, M., Chase, K., Sapp, E., Pfister, E., Sass, M., Yoder, J., Reeves, P., Pandey, R. K., Rajeev, K. G. et al. (2007). Therapeutic silencing of mutant huntingtin with siRNA attenuates striatal and cortical neuropathology and behavioral deficits. Proc Natl Aced Sci USA 104, 17204-9.

Everett, C. M. and Wood, N. W. (2004). Trinucleotide repeats and neurodegenerative disease. Brain 127, 2385-405.

Förster. (1948). Zwischenmolekulare Energiewanderung and Fluoreszenz. Annalen der Physik 437, 55-75.

Gabriel, D., Vernier, M., Pfeifer, M. J., Dasen, B., Tenaillon, L. and Bouhelal, R. (2003). High throughput screening technologies for direct cyclic AMP measurement. Assay Drug Dev Technol 1, 291-303.

Graham, R. K., Deng, Y., Slow, E. J., Haigh, B., Bissada, N., Lu, G., Pearson, J., Shehadeh, J., Bertram, L., Murphy, Z. et al. (2006). Cleavage at the caspase-6 site is required for neuronal dysfunction and degeneration due to mutant huntingtin. Cell 125, 1179-91.

Group, H. R. (1993). A novel gene containing a trinucleotide repeat that is expanded and unstable on Huntington's disease chromosomes. The Huntington's Disease Collaborative Research Group. Cell 72, 971-83.

Gubler, H. (2006). Methods for Statistical Analysis, Quality Assurance and Management of Primary HTS Data: Wiley-VCH.

Gusella, J. F., Wexler, N. S., Conneally, P. M., Naylor, S. L., Anderson, M. A., Tanzi, R. E., Watkins, P. C., Ottina, K., Wallace, M. R., Sakaguchi, A. Y. et al. (1983). A polymorphic DNA marker genetically linked to Huntington's disease. Nature 306, 234-8.

King, M. A., Hands, S., Hafiz, F., Mizushima, N., Tolkovsky, A. M. and Wyttenbach, A. (2008). Rapamycin inhibits polyglutamine aggregation independently of autophagy by reducing protein synthesis. Mol Pharmacol 73, 1052-63.

Ko, J., Ou, S. and Patterson, P. H. (2001). New anti-huntingtin monoclonal antibodies: implications for huntingtin conformation and its binding proteins. Brain Res Bull 56, 319-29.

Landles, C. and Bates, G. P. (2004). Huntingtin and the molecular pathogenesis of Huntington's disease. Fourth in molecular medicine review series. EMBO Rep 5, 958-63.

Lee, H. J., Hammond, D. N., Large, T. H. and Wainer, B. H. (1990). Immortalized young adult neurons from the septal region: generation and characterization. Brain Res Dev Brain Res 52, 219-28.

Li, H., Li, S. H., Johnston, H., Shelbourne, P. F. and Li, X. J. (2000). Amino-terminal fragments of mutant huntingtin show selective accumulation in striatal neurons and synaptic toxicity. Nat Genet 25, 385-9.

Li, P., Huey-Tubman, K. E., Gao, T., Li, X., West, A. P., Jr., Bennett, M. J. and Bjorkman, P. J. (2007). The structure of a polyQ-anti-polyQ complex reveals binding according to a linear lattice model. Nat Struct Mol Biol 14, 381-7.

Machida, Y., Okada, T., Kurosawa, M., Oyama, F., Ozawa, K. and Nukina, N. (2006). rAAV-mediated shRNA ameliorated neuropathology in Huntington disease model mouse. Biochem Biophys Res Commun 343, 190-7.

Mangiarini, L., Sathasivam, K., Seller, M., Cozens, B., Harper, A., Hetherington, C., Lawton, M., Trottier, Y., Lehrach, H., Davies, S. W. et al. (1996). Exon 1 of the HD gene with an expanded CAG repeat is sufficient to cause a progressive neurological phenotype in transgenic mice. Cell 87, 493-506.

Mathis, G. (1993). Rare earth cryptates and homogeneous fluoroimmunoassays with human sera. Clin Chem 39, 1953-9.

Menalled, L. B., Sison, J. D., Dragatsis, I., Zeitlin, S. and Chesselet, M. F. (2003). Time course of early motor and neuropathological anomalies in a knock-in mouse model of Huntington's disease with 140 CAG repeats. J Comp Neurol 465, 11-26.

Myers, R. H. (2004). Huntington's disease genetics. NeuroRx 1, 255-62.

Paganetti, P. A., Lis, M., Klafki, H. W. and Staufenbiel, M. (1996). Amyloid precursor protein truncated at any of the gamma-secretase sites is not cleaved to beta-amyloid. J Neurosci Res 46, 283-93.

Paganetti, P., Weiss, A., Trapp, M., Hammerl, I., Bleckmann, D., Bodner, R. A., Coven-Easter, S., Housman, D. E., Parker, C. N. (2009). Development of a method for the high-throughput quantification of cellular proteins. ChemBioChem 10, 1678-1688.

Perrin, V., Regulier, E., Abbas-Terki, T., Hassig, R., Brouillet, E., Aebischer, P., Luthi-Carter, R. and Deglon, N. (2007). Neuroprotection by Hsp104 and Hsp27 in lentiviral-based rat models of Huntington's disease. Mol Ther 15, 903-11.

Perutz, M. F., Johnson, T., Suzuki, M. and Finch, J. T. (1994). Glutamine repeats as polar zippers: their possible role in inherited neurodegenerative diseases. Proc Natl Acad Sci USA 91, 5355-8.

Perutz, M. F., Pope, B. J., Owen, D., Wanker, E. E. and Scherzinger, E. (2002). Aggregation of proteins with expanded glutamine and alanine repeats of the glutamine-rich and asparagine-rich domains of Sup35 and of the amyloid beta-peptide of amyloid plaques. Proc Natl Acad Sci USA 99, 5596-600.

Ravikumar, B., Duden, R. and Rubinsztein, D. C. (2002). Aggregate-prone proteins with polyglutamine and polyalanine expansions are degraded by autophagy. Hum Mol Genet 11, 1107-17.

Riddle, S. M., Vedvik, K. L., Hanson, G. T. and Vogel, K. W. (2006). Time-resolved fluorescence resonance energy transfer kinase assays using physiological protein substrates: applications of terbium-fluorescein and terbium-green fluorescent protein fluorescence resonance energy transfer pairs. Anal Biochem 356, 108-16.

Rodriguez-Lebron, E., Denovan-Wright, E. M., Nash, K., Lewin, A. S. and Mandel, R. J. (2005). Intrastriatal rAAV-mediated delivery of anti-huntingtin shRNAs induces partial reversal of disease progression in R6/1 Huntington's disease transgenic mice. Mol Ther 12, 618-33.

Ross, C. A. (2002). Polyglutamine pathogenesis: emergence of unifying mechanisms for Huntington's disease and related disorders. Neuron 35, 819-22.

Scherzinger, E., Sittler, A., Schweiger, K., Heiser, V., Lurz, R., Hasenbank, R., Bates, G. P., Lehrach, H. and Wanker, E. E. (1999). Self-assembly of polyglutamine-containing huntingtin fragments into amyloid-like fibrils: implications for Huntington's disease pathology. Proc Natl Acad Sci USA 96, 4604-9.

Sjogren, M., Davidsson, P., Wallin, A., Granerus, A. K., Grundstrom, E., Askmark, H., Vanmechelen, E. and Blennow, K. (2002). Decreased CSF-beta-amyloid 42 in Alzheimer's disease and amyotrophic lateral sclerosis may reflect mismetabolism of beta-amyloid induced by disparate mechanisms. Dement Geriatr Cogn Disord 13, 112-8.

Strozyk, D., Blennow, K., White, L. R. and Launer, L. J. (2003). CSF Abeta 42 levels correlate with amyloid-neuropathology in a population-based autopsy study. Neurology 60, 652-6.

van Roon-Mom, W. M., Reid, S. J., Jones, A. L., MacDonald, M. E., Faull, R. L. and Snell, R. G. (2002). Insoluble TATA-binding protein accumulation in Huntington's disease cortex. Brain Res Mol Brain Res 109, 1-10.

Varma, H., Voisine, C., DeMarco, C. T., Cattaneo, E., Lo, D. C., Hart, A. C. and Stockwell, B. R. (2007). Selective inhibitors of death in mutant huntingtin cells. Nat Chem Biol 3, 99-100.

Wang, Y. L., Liu, W., Wada, E., Murata, M., Wada, K. and Kanazawa, I. (2005). Clinico-pathological rescue of a model mouse of Huntington's disease by siRNA. Neurosci Res 53, 241-9.

Warby, S. C., Chan, E. Y., Metzler, M., Gan, L., Singaraja, R. R., Crocker, S. F., Robertson, H. A. and Hayden, M. R. (2005). Huntingtin phosphorylation on serine 421 is significantly reduced in the striatum and by polyglutamine expansion in vivo. Hum Mol Genet 14, 1569-77.

Weihofen, A., Lemberg, M. K., Friedmann, E., Rueeger, H., Schmitz, A., Paganetti, P., Rovelli, G. and Martoglio, B. (2003). Targeting presenilin-type aspartic protease signal peptide peptidase with gamma-secretase inhibitors. J Biol Chem 278, 16528-33.

Weiss, A., Klein, C., Woodman, B., Sathasivam, K., Bibel, M., Regulier, E., Bates, G. P. and Paganetti, P. (2008). Sensitive biochemical aggregate detection reveals aggregation onset before symptom development in cellular and murine models of Huntington's disease. J Neurochem 104, 846-58.

Wheeler, V. C., Auerbach, W., White, J. K., Srinidhi, J., Auerbach, A., Ryan, A., Duyao, M. P., Vrbanac, V., Weaver, M., Gusella, J. F. et al. (1999). Length-dependent gametic CAG repeat instability in the Huntington's disease knock-in mouse. Hum Mol Genet 8, 115-22.

White, J. K., Auerbach, W., Duyao, M. P., Vonsattel, J. P., Gusella, J. F., Joyner, A. L. and MacDonald, M. E. (1997). Huntingtin is required for neurogenesis and is not impaired by the Huntington's disease CAG expansion. Nat Genet 17, 404-10.

Yamamoto, A., Cremona, M. L. and Rothman, J. E. (2006). Autophagy-mediated clearance of huntingtin aggregates triggered by the insulin-signaling pathway. J Cell Biol 172, 719-31.

Yamamoto, A., Lucas, J. J. and Hen, R. (2000). Reversal of neuropathology and motor dysfunction in a conditional model of Huntington's disease. Cell 101, 57-66.

Young, A. B. (2003). Huntingtin in health and disease. J Clin Invest 111, 299-302.

Zhang, J. H., Chung, T. D. and Oldenburg, K. R. (1999). A Simple Statistical Parameter for Use in Evaluation and Validation of High Throughput Screening Assays. J Biomol Screen 4, 67-73.

Zourlidou, A., Gidalevitz, T., Kristiansen, M., Landles, C., Woodman, B., Wells, D. J., Latchman, D. S., de Belleroche, J., Tabrizi, S. J., Morimoto, R. I. et al. (2007). Hsp27 overexpression in the R6/2 mouse model of Huntington's disease: chronic neurodegeneration does not induce Hsp27 activation. Hum Mol Genet 16, 1078-90.

1. Use of a an immunoassay for measuring the amount of the soluble forms of the mutated polyQ protein in a biological sample, wherein the protein is selected from the group of huntingtin, androgen receptor, atrophin 1, ataxin 1, ataxin 2, ataxin 3, ataxin 7, TATA box binding protein or alpha1a voltage dependent calcium channel subunit; as a diagnostic tool, for monitoring disease progression or for monitoring the efficacy of treatment of the disease associated with the mutated polyQ form of the protein.

2. Use according to claim 1 wherein additionally the absolute or relative amount of the corresponding wild-type protein in the sample is measured in the immunoassay.

3. Use according to claim 1 or 2, wherein additionally the extent of post-translational modifications of the mutated protein is measured, such as cellular modifications of the expressed protein such as fragmentation by e.g proteolytical cleavage, phosphorylation, acetylation, ubiquitination, SUMOylation, lipid modification or other covalent modifications of the polypeptide backbone.

4. Use according to any of claims 1 to 3, wherein the immunoassay is a single step assay i.e. an immunoassay in which no separation or washing is necessary and which can preferably be run after a single biochemical handling.

5. Use according to any of claims 1 to 4, wherein the immunoassay detection technology is based on time-resolved Förster resonance energy transfer or electro-chemiluminescence.

6. Use according to claim 5, wherein the immunoassay detection technology is time-resolved Förster resonance energy transfer

7. Use according to claim 5, wherein the immunoassay comprises the following steps
a) contacting the biological sample with a first antibody labeled with: a lanthanoide ion cryptate (such as europium or terbium cryptate) and a second antibody labeled with a fluorophore suited for detecting the lanthanide emitted signal, where one of the antibodies is specific for the polyQ part of the mutant protein and the other antibody is specific for a different part of the mutant huntingtin protein and
b) quantifying the amount of mutant PolyQ protein in the sample by measuring the fluorescence from the fluorophore by time-resolved Förster Resonance Energy Transfer.

8. Use according to claim 5, wherein in addition the (relative) amount of the corresponding wild-type protein in the sample is measured in the biological sample by additionally contacting it with a third antibody specific for the wild-type form of the protein and labeled with a different fluorophore suited for detecting the lanthanide emitted signal.

9. Use according to any of the previous claims, wherein the polyQ-protein is polyQ-huntingtin.

10. Use according to any of the preceding claims, wherein the biological sample is derived from the brain, from blood, from muscle or heart or derived from peripheral tissue such as skin or hair.

11. An immunoassay for measuring the amount of the soluble forms of mutated (expanded polyQ) protein in a biological sample, wherein the protein is selected from the group of huntingtin, androgen receptor, atrophin 1, ataxin 1, ataxin 2, ataxin 3, ataxin 7, TATA box binding protein or alpha1a voltage dependent calcium channel subunit

12. An immunoassay according to claim 11 wherein additionally the absolute or relative amount of the corresponding wild-type protein in the sample is measured in the immunoassay.

13. An immunoassay according to claim 11 or 12, wherein additionally the extent of post-translational modifications of the mutated protein is measured, such as cellular modifications of the expressed protein such as fragmentation by e.g proteolytical cleavage, phosphorylation, acetylation, ubiquitination, SUMOylation, lipid modification or other covalent modifications of the polypeptide backbone.

14. An immunoassay according to any of claims 11 to 13, wherein the immunoassay is a single step assay, i.e. an immunoassay in which no separation or washing is necessary and which can preferably be run after a single biochemical handling.

15. An immunoassay according to any of claims 11 to 14, wherein the immunoassay detection technology is based on time-resolved Förster resonance energy transfer or electrochemiluminescence.

16. An immunoassay according to claim 15, wherein the immunoassay detection technology is time-resolved Förster resonance energy transfer

17. An immunoassay according to claim 15, which comprises the following steps
a) contacting the biological sample with a first antibody labeled with: a lanthanoide ion cryptate (such as europium or terbium cryptate) and a second antibody labeled with a fluorophore suited for detecting the lanthanide emitted signal, where one of the antibodies is specific for the polyQ part of the mutant protein and the other antibody is specific for a different part of the mutant huntingtin protein and
b) quantifying the amount of mutant PolyQ protein in the sample by measuring the fluorescence from the fluorophore by time-resolved Förster Resonance Energy Transfer.

18. An immunoassay according to claim 17, wherein in addition the (relative) amount of the corresponding wild-type protein in the sample is measured in the biological sample by additionally contacting it with a third antibody specific for the wild-type form of the protein and labeled with a different fluorophore suited for detecting the lanthanide emitted signal.

19. An immunoassay according to any of the previous claims, wherein the polyQ-protein is polyQ-huntingtin.

20. An immunoassay according to any of the preceding claims, wherein the biological sample is derived from the brain, from blood, from muscle or heart or derived from peripheral tissue such as skin or hair.
